(19) United States
(12) Patent Application Publication
(10) Pub. No.: US 2022/0284794 A1
(43) Pub. Date: Sep. 8, 2022

(54) SYSTEMS AND METHODS FOR INCREASED ACCURACY FOR TRACKING HYGIENE COMPLIANCE

(71) Applicant: GOJO Industries, Inc., Akron, OH (US)

(72) Inventor: Jackson W. Wegelin, Stow, OH (US)

(21) Appl. No.: 17/626,144

(22) PCT Filed: Feb. 4, 2021
(86) PCT No.: PCT/US2020/043438

§ 371 Date: Jan. 11, 2022

Related U.S. Application Data
(60) Provisional application No. 62/878,921, filed on Jul. 26, 2019.

(51) Int. Cl. G08B 21/24; G01S 13/46; G01S 13/68; G01S 5/04

(52) U.S. Cl. CPC G08B 21/245; G01S 13/46; G01S 13/68; G01S 5/04; G01S 2013/468

(57) ABSTRACT

An exemplary compliance monitoring system includes a dispenser for dispensing soap or sanitizer, a one people counter for detecting the presence of one or more persons or assets, at least one badge and at least one locatee device. The locatee device includes an antenna and transmitting circuitry for transmitting signals. At least one of the dispenser, the people counter, and the one or more badges include a locatee device. A locator device is also provided. The locatee device circuitry includes wake-up circuitry for instructing the locatee device circuitry to transmit one or more signals, at least two antennae, triangulation based measurement circuitry to determine the angle of arrival of the one or more signals, and wake-up circuitry for waking up the triangulation based measurement circuitry. The wake-up circuitry causes the triangulation based measurement to listen for the one or more signals from the at least one locatee device.

### RELATED APPLICATIONS

This application claims the benefits of, and priority to, U.S. Provisional Patent Application 62/878,921 titled SYSTEMS AND METHODS FOR INCREASED ACCURACY FOR TRACKING HYGIENE COMPLIANCE, which was filed on Jul. 26, 2019, and which is incorporated by reference herein in its entirety.

### TECHNICAL FIELD

The present invention generally relates to systems and methods for increased accuracy in tracking hygiene compliance. Particularly, the present invention relates to use of triangulation systems and methods for more accurately determining locations of persons and/or assets in hygiene compliance monitoring systems.

### BACKGROUND OF THE INVENTION

Compliance monitoring systems are designed to provide companies or institutions with knowledge relating to compliance with selected hand hygiene policies and/or asset cleaning policies. Such companies and institutions include, for example, hospitals, food service industries, clinics, and the like. Several situations occur in which it is desirable to know whether a person (or the number of people and/or other moving objects/assets) is passing through or occupying an area, the time a person passes through an area, or any other type of information for monitoring the movement of one or more people through an area. For example, a hospital administrator, for hygiene compliance purposes, may want to know the number of people that entered a room having a hand soap or sanitizer dispenser, such that a comparison can be made between the number of people that entered the room (i.e., number of opportunities for use of the dispenser) and the number of actual uses of the hand soap or sanitizer dispenser. In addition, it would be helpful to be able to precisely locate people to determine with more accuracy which person obtained a dose of hand sanitizer, washed their hands, or which person/asset entered an area triggering an hand-hygiene opportunity.

The monitoring of hand washing/sanitizing by individuals who are identified by electronic badges or data tags and then associating the badges or tags and individuals with the use of hygiene dispensers is known in the art. In addition, there are some automated system providers for hygiene compliance monitoring systems that use people counters with wireless communication circuitry for transmitting the location of people. The dispensers, badges, and/or people counters transmits dispense events, user identification, and sometimes location to a central computer to record and analyze the usage data.

While such systems perform well when a single person is in the room or area, they may not perform well when several people are located in the same room or in close proximity the dispensers.

### SUMMARY

Exemplary embodiments of compliance monitoring systems, dispensers and people counters are disclosed herein. An exemplary compliance monitoring system includes at least one dispenser for dispensing soap or sanitizer, at least one people counter for detecting the presence of one or more persons or assets, at least one badge associated with a person or asset, and at least one locatee device. The locatee device includes at least one antenna and transmitting circuitry for transmitting one or more signals. At least one of the at least one dispenser, the at least one people counter, and the one or more badges include a locatee device. A locator device is also provided. The locatee device circuitry includes wake-up circuitry for instructing the locatee device circuitry to transmit one or more signals, at least two antennae, triangulation based measurement circuitry to determine the angle of arrival of the one or more signals, and wake-up circuitry for waking up the triangulation based measurement circuitry. The wake-up circuitry causes the triangulation based measurement to listen for the one or more signals from the at least one locatee device.

An exemplary dispenser for dispensing soap or sanitizer includes the dispenser having a housing, a processor, a sensor for sensing an object, a holder for holding a container of soap or sanitizer, a pump in fluid communication with the container, an actuator for causing the pump to dispense a dose of fluid, communication circuitry for wireless communication, triangulation based locator circuitry, two or more antennae, and wake up circuitry for causing triangulation based locator circuitry to listen for signals from one or more locatee devices and determine a location of at least one of the locatee devices.

An exemplary people counter for detecting the presence of a person or object includes a housing, a processor, a sensor for sensing the presence of a person or object, communication circuitry for wireless communication, triangulation based locator circuitry, two or more antennae, and wake up circuitry for causing triangulation based locator circuitry to listen for signals from one or more locatee devices. Upon detection of signals from one or more locatee devices, the triangulation based locator circuitry determines an angle of arrival of one or more signals.

Another exemplary people counter for detecting the presence of a person or object includes a housing, a processor, a sensor for sensing the presence of a person or object, and communication circuitry for wireless communication. Upon detecting the presence of a person or object, the processor causes communication circuitry to broadcast a wake up wake-up signal that causes one or more locatee devices to wake up and transmit one or more signals. Triangulation based measurement circuitry for determining the location of one or more locatee devices is also included. The triangulation based measurement circuitry having two or more antennae.

### BRIEF DESCRIPTION OF THE DRAWINGS

These and other features and advantages of the present invention will become better understood with regard to the following description and accompanying drawings in which:

FIG. 1 is illustrative of an exemplary compliance monitoring system having triangulation based location detection;

FIG. 2 is a simplified illustration of triangulation based location detection.

FIG. 3 is a schematic block diagram of an exemplary embodiment of a locator device;

FIG. 4 a schematic block diagram of an exemplary embodiment of a locatee device; and

FIG. 5 is another schematic block diagram of an exemplary embodiment of method for locating a person, device, or asset with respect to a locate tour device.

### DETAILED DESCRIPTION

The following includes definitions of exemplary terms used throughout the disclosure. Both singular and plural forms of all terms fall within each meaning. Except where noted otherwise, capitalized and non-capitalized forms of all terms fall within each meaning:

“Circuit communication” as used herein indicates a communicative relationship between devices. Direct electrical, electromagnetic and optical connections and indirect electrical, electromagnetic and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers or satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices not directly connected to each other, but both capable of interfacing with a third device, such as, for example, a CPU, are in circuit communication. Circuit communication includes providing power to one or more devices. For example, a processor may be in circuit communication with one or more batteries, indicating that the batteries provide power to the processor.

Also, as used herein, voltages and values representing digitized voltages are considered to be equivalent for the purposes of this application, and thus the term “voltage” as used herein refers to either a signal, or a value in a processor representing a signal, or a value in a processor determined from a value representing a signal.

“Signal”, as used herein includes, but is not limited to one or more electrical signals, power signals, analog or digital signals, one or more computer instructions, a bit or bit stream, or the like.

“Logic,” synonymous with “circuit” as used herein includes, but is not limited to hardware, firmware, software and/or combinations of each to perform a function(s) or an action(s). For example, based on a desired application or needs, logic may include a software controlled microprocessor or microcontroller, discrete logic, such as an application specific integrated circuit (ASIC) or other programmed logic device. Logic may also be fully embodied as software. The circuits identified and described herein may have many different configurations to perform the desired functions.

Any values identified in the detailed description are exemplary and they are determined as needed for a particular dispenser and/or refill design. Accordingly, the inventive concepts disclosed and claimed herein are not limited to the particular values or ranges of values used to describe the embodiments disclosed herein.

Power connection as used herein indicates a power relationship between devices. Direct electrical connections as well as inductive power connections are examples of circuit communication.

The term people counter as used herein does not require the system to locate and count people. In many cases, the people counter locates a badge or tag. Typically, the badge or tag is associated with, and/or secured to a person. In some embodiments, the badge or tag is associated with an asset. Accordingly, the people counter may detect an asset that is associated with the badge or tag, a badge or tag attached to a person, or a person.

In addition, the term “badge” or “tag” as used herein may be a stand-alone device, or may be an integrated device, such as, for example, a smart phone, tablet, iPad, or the like. In some embodiments, the integrated device may include application software so that the device performs any of the required functions described herein.

In some embodiments, the people counter may detect a signal, such as, for example, a BlueTooth signal emitted form that badge, smart phone, tablet, iPad or other device. In some embodiments, the people counter may cause the badge or other device to emit a signal detectable by the people counter.

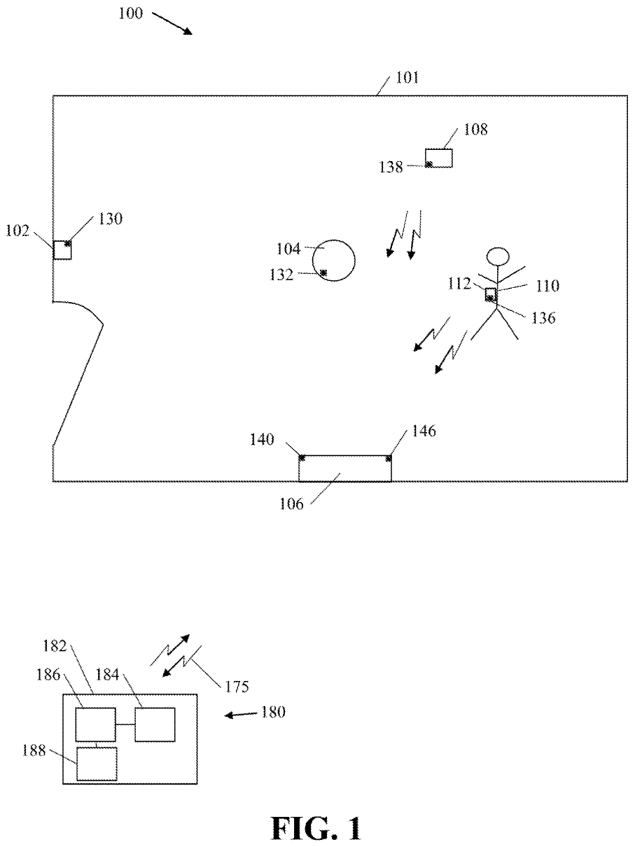

FIG. 1 illustrates an exemplary embodiment of compliance monitoring system 100. The exemplary compliance monitoring system 100 covers one or more rooms 101. Only one room 101 is shown, however many rooms may be incorporated in the system. The rooms may be part of a facility, such as, for example, a hospital, a restaurant, or the like, where it is important for people to maintain proper hand-hygiene, or ensure an asset is properly cleaned and/or easily locatable. Compliance monitoring system 100 includes a plurality of fixed dispensers 102 (only 1 is shown for purposes of clarity), a plurality of badges 102 (only 1 is shown for clarity), one or more gateways 106 (in some instances gateways 106 are not required) and an optional compliance monitoring station 180. In addition, in some embodiments, compliance monitoring system 100 includes one or more moveable assets 108. In the exemplary embodiment, the moveable asset 108 is a dispenser. In some embodiments, the moveable asset is a piece of equipment used for a patient. In some embodiments, the moveable asset is a piece of equipment used for cleaning. In some embodiments, the moveable asset is a piece of equipment used for moving equipment or food.

Compliance monitoring station 108 may be, for example, a computer having software/logic for determining, inter alia, one or more compliance rates for individuals as a function of, for example, the number of dispenses of soap or sanitizer delivered to an individual divided by the number of opportunities that the person had washed or sanitized their hands.

Dispenser 102 may be any type of dispenser, such as, for example, a touch free dispenser. Exemplary touch-fee dispensers are shown and described in U.S. Pat. No. 7,837,066 titled Electronically Keyed Dispensing System And Related Methods Utilizing Near Field Response; U.S. Pat. No. 9,172,266 title Power Systems For Touch Free Dispensers and Refill Units Containing a Power Source; U.S. Pat. No. 7,909,209 titled Apparatus for Hands-Free Dispensing of a Measured Quantity of Material; U.S. Pat. No. 7,611,030 titled Apparatus for Hans-Free Dispensing of a Measured Quantity of Material; U.S. Pat. No. 7,621,426 titled Electronically Keyed Dispensing Systems and Related Methods Utilizing Near Field Response; and U.S. Pat. No. 8,960,498 titled Touch-Free Dispenser with Single Cell Operation and Battery Banking; U.S. Pat. No. 9,984,553 titled Hygiene Compliance Module; all which are incorporated herein by reference. In some embodiments, the dispenser may be a manually operated dispenser. In such a dispenser, a user manually causes the dispenser to dispense product. The user may manually cause the dispenser to dispense product by, for example, pressing a push-bar; pulling a lever; pushing a lever; stepping on a foot activated pump; and the like.

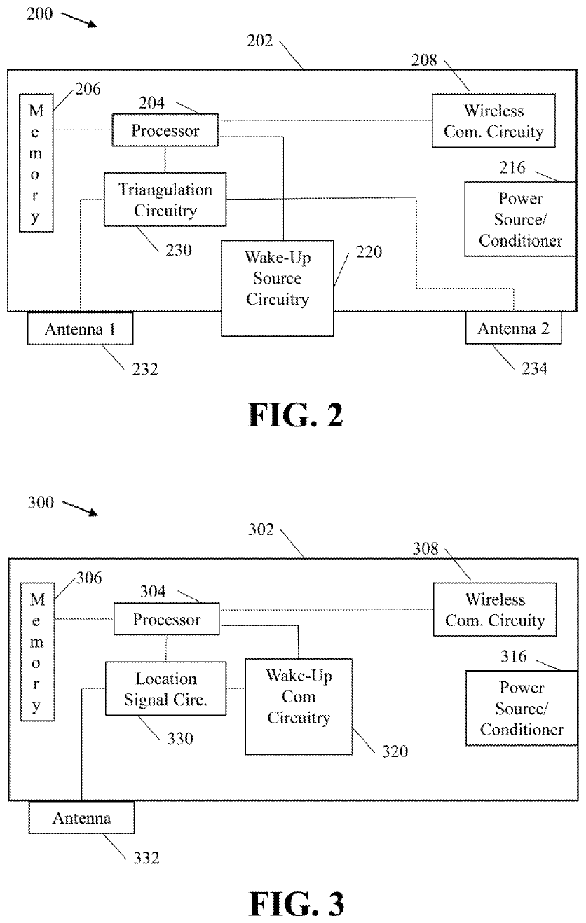

The compliance monitoring system 100 includes locator devices 200 (FIG. 2) and locatee devices 300 (FIG. 3). The locator devices 200 obtain accurate locational data for the locatee devices 300. The accurate locational data is obtained by use of triangulation based measurements, described in detail below.

In this exemplary embodiment, dispenser 102 is a fixed dispenser, i.e. dispenser 102 is mounted to a fixed location, such as, for example, a wall and does not move. In this exemplary embodiment, dispenser 102 has a “locator” device 130 and contains the locator circuitry shown in FIG. 2. However in some embodiments, dispenser 102 may be a “locatee” device and contain the locatee circuitry shown in FIG. 3. Typically, the system includes one or more locator devices depending on the particulars of the area(s) being monitored. In some embodiments, there is a locator device in each room or area. In some embodiments, for larger rooms or larger areas, there may be more than one locator device is in a room or area. In some embodiments, additional locator devices are required due to one or more factors that limit the required wireless communications described herein, such as, for example, walls or equipment.

The locator circuitry 200 may be a stand-alone device or may be located in one or more of a fixed dispenser 102, a people counter 104, a hub, a gateway 106 and the like. Preferably the locator circuitry 200 is located in a stationary device. In this exemplary embodiment, people counter 104 includes an optional locator device 132. In this exemplary embodiment, gateway 106 includes an optional locator device 140. In most embodiments, only one locator device is required in an area, the inclusion of the optional locator devices is for illustrating optional devices/locations for locator devices. In some embodiments, stationary devices such as, for example, gateway 106 includes an optional locatee device 149. Similarly, in some embodiments, people counter 104 includes an optional locatee device (not shown). Including the optional locatee devices in various components of the system may allow a locator device to more easily determine the location of one locatee device with respect to another.

In some embodiments, dispenser 102 is equipped with circuitry described herein with respect to locatee devices 300 and include locatee device circuitry. In this exemplary embodiment, dispenser 102 is equipped with the circuitry described herein with respect to locator device 200 and include locate device circuitry 200.

In addition to circuitry required or used for their primary functions, locator devices include locator device circuitry 200. Locator device circuitry 200 may be located on a printed circuit board 202. In some embodiments, the components are located on a single printed circuit board. In some embodiments, the components are located on one or more printed circuit boards. In some embodiments, the components are located on one or more printed circuit board and/or are separately mounted to the device housing and placed in circuit communication with the other circuitry described herein.

In this exemplary embodiment, locator device 200 includes a processor 204, memory 206, wireless communication circuitry 208, triangulation circuitry 230, wake-up source circuitry 220, two or more antennae 232, 234, and a power source/conditioner 216. The components are connected to one another and/or are otherwise configured as needed in a typical manner. In some embodiments, the locator circuitry 200 is connected to a power source/conditioner of a specific device (not shown) that the circuitry is located in. For example, locator circuitry 200 may be powered from the batteries in a dispenser 102. In some embodiments, locator device 200 is a separate device and is placed proximate one of the other devices, such as, for example, a gateway 106, or a dispenser 102. In some embodiments, the above identified components may be shared with the device that is housing the locator device circuitry. For example, in the exemplary embodiment of FIG. 1, dispenser 102 contains locator device 200 circuitry and processor 204 and memory 206 may be the same processor and memory that causes the dispenser to function as intended.

Wireless communication circuitry 208 may include longer range communication circuitry, such as, for example, a radio frequency (“RF”) transceiver; in some embodiments, the wireless communications circuitry 208 includes near field communications such as, for example, BlueTooth® transmitter/receiver. Other types of wireless circuitry, such as, for example, BLE, Infrared (“IR”) or ANT®, may also be used. Wireless communication circuitry 208 may be used to communicate with one or more of the compliance monitoring station 180, people counter 104, badge 112, gateway 106 and/or mobile asset 108.

Locator device 200 also includes wake-up source circuitry 220. Wake-up source circuitry 220 is used to wake-up the triangulation circuitry 230. In some embodiments, wake-up source circuitry 220 includes one or more of an IR receiver, an RF receiver, a motion detector, an infrared detector, a heat source detector, a sound detector, or the like. The wake-up source circuitry 220 is a low power device. A low power device allows for the wake-up source circuitry to be on, or intermittently on, for long periods of time without drawing a significant amount of power, which is desirable when power source/conditioner 216 includes one or more batteries to power the locator device 200. Him and his

When wake-up source circuitry 220 detects the presence of a person, asset or badge, triangulation circuitry 230 wakes up and begins to determine a location of one or more locatee devices 300. Triangulation circuitry 230 utilizes two or more antennae 232, 234 to receive signals from the locatee device 300.

In some embodiments, wake-up source circuitry 220 is embodied in software or logic and the locator device receives the “wake-up” signal via the wireless communication circuitry 208. Thus, for example, if a person 110 having a badge 112 enters a room, people counter 104 may detect the presence of the person 110 and broadcast a communication on its wireless communication circuitry. Dispenser 102, which in this exemplary embodiment is a locator device 200 and therefore contains triangulation circuitry 230, receives the communication and causes the triangulation circuitry 230 to listen for an signal.

When a wake up signal is detected, locator device 200 triggers triangulation circuitry 230 to power up so that it can receive one or more signals, for example, one or more Impulse Radio Ultra-Wideband (“IR-UWB”) signals from the location signal circuitry 330 of the locatee device 300 (described in detail with respect to FIG. 3). In this exemplary embodiment, triangulation circuitry 230 receives the one or more IR-UWB signals on each of each of the two or more antennae 232, 234 and uses those IR-UWB signals to determine the location of the locatee device 200. Triangulation circuitry 230 uses angle of arrival “AOA” methods for determining the location of the locatee device 200 from receipt of one or more signals on the two or more antennae 232, 234. Four exemplary AOA methods are time-of-flight (“TOF”), Time Difference of Arrival (“TDOA”), Phase Difference of Arrival (“PDOA”) and TDOA/PDOA hybrid. More detailed information on calculating the AOA may be found in Angle of Arrival Estimation Using Decawave DW1000 Integrated Circuits, authored by Igor Dotlic, Andrew Connell, Hang Ma, Jeff Clancy, Michael McLaughlin, which may be found at https://www.decawave.com/sites/default/files/angle of arrival estimation using dw 1000 onlin e.pdf, and U.S. Pat. No. 10,056,993 titled Angle of arrival using reduced number of receivers. Both of which are incorporated herein by reference in their entirety.

Locatee device 300 may include a processor 304, memory 306, wake-up communication circuitry 320, location signal circuitry 330, one or more antennae 332, and a power source/conditioner 316. In some embodiments, optional wireless communication circuitry 308 is also included. The components are connected to one another and/or are otherwise configured in a typical manner. In some embodiments, the locatee device 300 is connected to a power source/conditioner of another device (not shown) that the locatee device 300 is located in or located near. Preferably locatee device 300 is located within another device, however, in some embodiments, locatee device 300 is a separated device and placed proximate one of the other devices, such as, for example, a gateway, or a dispenser. When locatee device 300 is in another device, the components identified above may be components of the other device. For example, the processor and memory, may be part of a badge and used for other purposes as well.

Wake-up communication circuitry 320 is used to wake-up the location signal circuitry 230. In some embodiments, wake-up communication circuitry 320 includes one or more of an IR receive, an RF receiver, a motion detector, an IR detector, or the like. The wake-up communication circuitry 320 is a low power device. A low power device allows for the wake-up source circuitry to be on, or intermittently on, for longer periods of time without drawing a significant amount of power, which is desirable when power source/conditioner 316 includes one or more batteries to power the locatee device 300.

Wake-up source circuitry 320 detects the presence of a wake-up signal. The wake-up signal may be received by wake-up communication circuitry 320 directly. In some embodiments, the wake-up signal is received via the wireless communication circuitry 308 in locator device 300. When locatee device 300 receives a “wake-up” signal, location signal circuitry 330 transmits one or more signals from the one or more antenna 332 to the two or more antennae, 232, 234 in the locator device 200. In some embodiments, wake-up circuitry 320 may be in software and/or logic and wireless communication circuitry 308 may be used to receive and/or transmit the “wake-up” signal.

In the embodiments that include wireless communication circuitry 308, the wireless communication circuitry 308 may include longer range communication circuitry, such as, for example, a radio frequency (“RF”) transceiver. In some embodiments, the wireless communications circuitry 308 includes near field communications such as, for example, BlueTooth® transmitter/receiver. Other types of wireless circuitry, such as, for example, BLE, Infrared (“IR”) or ANT®, may also be used. In some embodiments, near field communications and longer range communication circuitry are used in wireless communication circuitry 308.

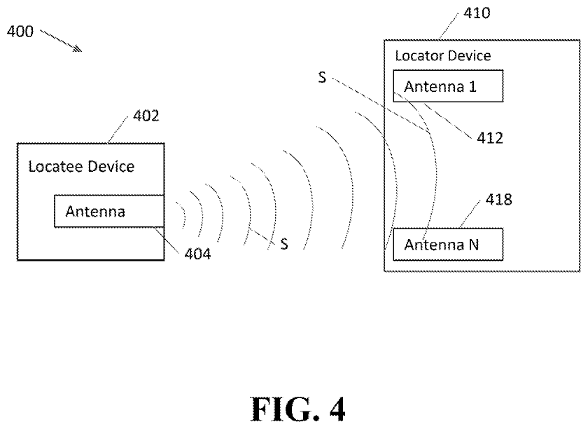

FIG. 4 is an exemplary embodiment of a portion of a system 400 for determining the location of one or more people and/or one or more moveable objects in a hand-hygiene compliance system. In this exemplary embodiment, locatee device 402 wakes up and transmits an Impulse Radio Ultra-Wideband (“IR-UWB”) pulse signals S. In most embodiments, locatee device 402 does not continuously transmit IR-UWB pulses, but rather only transmits pulses when a wake-up signal is received by the locatee device 402 as described above. Locator device 410 also receives a wake-up signal and wakes up to receive the IR-UWB pulse signals S on two or more antennae, antenna 412 and antenna 418. Locator device 410 uses the received IR-UWB signals S to determine AOA and the location of the locatee device 402. In some embodiments, circuitry (not shown), which may include logic and/or hardware, in locator device 410 determines time-of-flight (“TOF”) of the IR-UWB pulse signal S to each of the antennae 412, 418. The difference between two measured times of flight valves may be used to estimate the AOA which is used to determine the location of the locatee device 402. In some embodiments, circuitry (not shown), which may include logic and/or hardware, in locator device 410 detects time difference of arrival (“TDOA”) of the IR-UWB pulse signals S to each of the antennae 412, 418. The difference between two receiving “time stamps” of the same frame may be used to estimate the AOA which is used to determine the location of the locatee device 402. In some embodiments, circuitry (not shown), which may include logic and/or hardware, in locator device 410 detects phase difference of arrival (“PDOA”) of the IR-UWB pulse signals S to each of the antennae 412, 418. The difference between the two or more phases of the received IR-UWB pulse signals S may be used to estimate the AOA which is used to determine the location of the locatee device 402. In some embodiments, circuitry (not shown), which may include logic and/or hardware, in locator device 410 uses a TDOA/PDOA hybrid where, for distancers between antennae above half-wavelength, TDOA is used to select one of a pre-defined AOS interval and PDOA is used to get at an AOA estimate within the selected interval to estimate the AOA which is used to determine the location of the locatee device 402. Using triangulation based circuitry allows the locator device 410 to accurately determine the location of the locatee device 402. Accordingly, if there are several people or moveable assets that contain locatee circuitry, the locator device can determine the location of each with near certainty.

In some embodiments, the system knows the exact location of the dispensers or other devices that are being monitored to determine whether an individual uses the device. In some embodiments, the device, such as, for example, a dispenser, includes a locatee circuitry even though the dispenser does not move. Because the system knows the locations of all of the locatee devices and knows who or what is associated with the locatee device, the system can accurately determine which person received a dose of fluid from the dispenser. In addition, the system can accurately determine who/what is in a selected area at any given time.

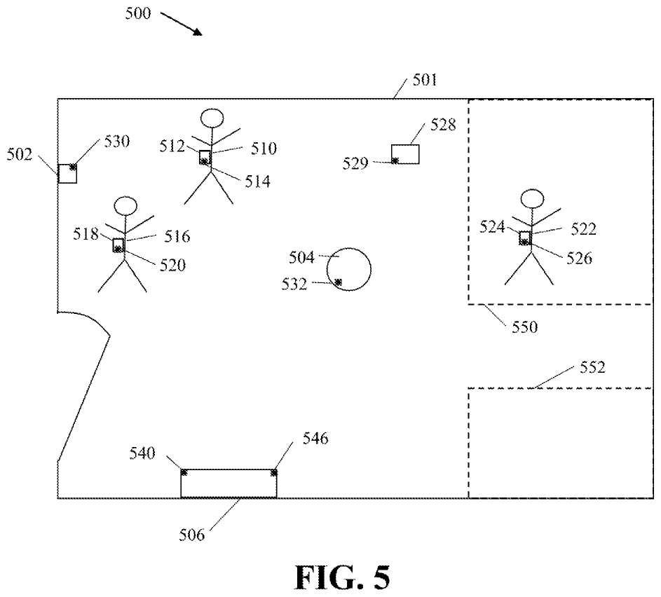

FIG. 5 illustrates another exemplary embodiment of an improved compliance monitoring system 500 that uses triangulation based circuitry to accurately determine accurate locations of persons/objects. The accurate locations may be used to more accurately determine when a person or object triggers an opportunity for hygiene event. A hygiene event may be washing ones hands, sanitizing ones hands, cleaning or sanitizing of the object, or the like.

Compliance monitoring system 500 illustrates a single room 501, however, in many embodiments, the system 500 may include many rooms. In some embodiments, the system may include hallways, nursing stations, and/or other areas where hand hygiene opportunities occur and/or where hand washing or sanitizing stations are located. In some embodiments, compliance monitoring system 500 is in a restaurant or food processing plant. In this exemplary embodiment, room 501 includes a dispenser 502, a people counter 504, and a gateway 506. In addition, several people 510, 516, 522 and a moveable asset 528 are presently in room 501. In some exemplary embodiments, each person 510, 516, 522 and/or asset 528 has a badge associated therewith. For example, person 510 has a badge 512, person 516 has a badge 518 and person 522 has a badge 524 associated therewith. Similarly, movable asset 528 has a badge 529. As described above, the term “badge” should be construed broadly to include any identification device with the capabilities described herein, such as, for example, a tag, a bracelet, a personal data device, and the like. Each badge contains locatee circuitry 200. In this exemplary embodiment, room 501 contains a patient zone 550. Patient zone 550 is an area near a patient (not shown) whereby a person in patient zone 550 may be assumed to be treating or attending to a patient. Room 501 also includes a contamination area 552, such as, for example, a bathroom 552 and a person entering or leaving the contamination area 552 may be assumed to need to perform a hand-hygiene function.

When one or more people 510, 516, 522 enter room 501, people counter 504 detects the presence of the one or more people 510, 516, 522 and broadcasts a wake-up signal. All of the badges 518, 512, 524, assets 528, dispenser 502, and the like, that contain locatee circuitry 300, wake up and broadcast location signals. Locator circuitry 200, which in this exemplary embodiment is located in people counter 504, determines the locations of each of the devices that broadcast the location signals. In some exemplary embodiments, gateway 506 includes an optional locator device 540. In most embodiments, only one locator device is required in an area, the inclusion of the optional locator devices is for illustrating optional devices/locations for locator devices. In some embodiments, stationary devices such as, for example, gateway 506 includes an optional locatee device 546.

Exemplary embodiments of people counters are more fully described in U.S. Pat. No. 1,057,709, which is titled Systems for Providing Condition-Based Data From a User Interactive Device, which is incorporated herein by reference in its entirety; and U.S. Non-Provisional patent application Ser. No. 16/274,597, which was filed on Feb. 13, 2019 and which is titled Module People Counter and which is also incorporated herein by reference in its entirety.

In some exemplary embodiments, when dispenser 502 has a dispense event, it broadcasts a wake-up signal. All of the badges 518, 512, 524, assets 528, dispenser 502, and the like, that contain locatee circuitry 300, wake up and broadcast a location signal. Locator circuitry 200 in people counter 504 wakes up and locator circuitry 200 determines the location of the one or more of the dispenser 502, badges 518, 512, 524, and the asset 528. Locator circuitry 200 utilizes triangulation based circuitry to determine the locations very accurately. In an exemplary embodiment, locator circuitry 200 is used to determine that person 516 is located closest to dispenser 502 that just dispensed a dose of product. The compliance monitoring system (not shown) uses the location information to associate the dispense event with person 516. If for example, asset 528, which in this case is a movable dispenser, broadcasts a dispense event, locator circuitry 200 in people counter 504 determines person 522 is closest to the dispenser and associates the dispense event with person 522.

An exemplary compliance monitoring system is shown and disclosed in U.S. patent application Ser. No. 14/789,595, titled METHODS AND SYSTEMS FOR IMPROVING HAND HYGIENE, which is incorporated herein by reference. A simplified version of a compliance monitoring system uses the number of “opportunities” for a person to perform a hand hygiene event and the number of dispenses dispensed to the person to determine a compliance rate for that person. For example, if the system determines that a person enters an area, such as, for example, a room, a patient zone, o contaminant zone, or the like, the system may determine that the entry triggered an opportunity for that person and associates that opportunity with the person. If the person washes there hand and receives a dispense of soap, or receives a dispense of hand sanitizer, within selected time periods before or after an “opportunity” is triggered, the person is associated with a credit for a hand hygiene events. Periodically, a “compliance rate” or “compliance metric” is determined as a function of the credits for hand hygiene events and the number of opportunities. The function may be, for example, simply dividing the number of credits for hand hygiene events by the number of opportunities.

In this exemplary embodiment, people counter 504 may periodically broadcast a wake-up signal and determine the locations of the people/objects that have locatee circuitry 200 associated therewith. In some embodiments, the wake-up signal is broadcast every couple of seconds. In some embodiments, the signal is broadcast more frequently. In some embodiments, the signal is broadcast less frequently. In addition, in some embodiments, the signal may be broadcast more frequently if there are more people present in the area. Each time a wake-up signal is broadcast, locatee devices transmit a location signal and locator device(s) determine the locations of the locatee devices.

In this exemplary embodiment, people counter 504 may determine that person 522 is located in a patient zone 550 and therefore has triggered an opportunity. People counter 504 may determine that asset 528 is a dispenser and that a dispense event has occurred. Because a dispense event occurred, and because person 522 was closest to the asset 528, person 522 is credited with a hand-hygiene event. People counter 504 may determine that no one was near contamination zone 552. The dispense events, locations, time stamps, and opportunities, may be transmitted to gateway 506 and sent to a master station (not shown). After broadcasting the location signal, locatee circuitry 300 goes back to “sleep”. After determining the locations of all locatee devices, the locator circuitry 200 goes back to sleep.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. It is not the intention of the applicant to restrict or in any way limit the scope of the appended claims to such detail. Unless expressly excluded herein, all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order in which the steps are presented to be construed as required or necessary unless expressly so stated.

I/We claim:

1. A soap or sanitizer dispenser comprising:
a housing;
a processor;
a sensor for sensing an object;
a container for holding soap or sanitizer;
a pump in fluid communication with the container;
an actuator for causing the pump to dispense a dose of fluid;
communication circuitry for wireless communication;
triangulation based locator circuitry;
two or more antennae for receiving signals from one or more locatee devices; and
circuitry for causing triangulation based locator circuitry to listen for signals from the one or more locatee devices and circuitry to determine a location of at least one of the locatee devices.

2. The soap or sanitizer dispenser of claim 1 wherein the triangulation based locator circuitry calculates an angle of arrival from one or more signals received by the two or more antennae.

3. The soap or sanitizer dispenser of claim 2 wherein the angle of arrival is a function of two or more measured times of flight values of the one or more signals.

4. The soap or sanitizer dispenser of claim 2 wherein the angle of arrival is a function of the difference between two or more timestamps of the one or more signals.

5. The soap or sanitizer dispenser of claim 2 wherein the angle of arrival is a function of phase differences of the one or more signals.

6. The soap or sanitizer dispenser of claim 1 wherein the triangulation based locator circuitry determines what locatee devices is closest to the dispenser.

7. The soap or sanitizer dispenser of claim 1 further comprising wake-up circuitry for causing the triangulation circuitry to listen for signals.

8. A compliance monitoring system comprising:
at least one dispenser for dispensing soap or sanitizer;
at least one people counter for detecting the presence of one or more persons or assets;
at least one badge associated with a person or asset;
at least one locatee device;
wherein the locatee device comprises
at least one antenna;
transmitting circuitry for transmitting one or more signals
wherein at least one of the at least one dispenser, the at least one people counter, and the one or more badges include a locatee device;
a locator device;
wherein the locatee device circuitry comprises
wake-up circuitry for instructing the locatee device circuitry to transmit one or more signals;
at least two antennae;
triangulation based measurement circuitry to determine the angle of arrival of the one or more signals; and
wake-up circuitry for waking up the triangulation based measurement circuitry;
wherein the wake-up circuitry causes the triangulation based measurement to listen for the one or more signals from the at least one locatee device.

9. The compliance monitoring system of claim 8 wherein the angle of arrival is a function of two or more measured times of flight values of the one or more signals.

10. The compliance monitoring system of claim 8 wherein the angle of arrival is a function of the difference between two or more timestamps of the one or more signals.

11. The compliance monitoring system of claim 8 wherein the angle of arrival is a function of phase differences of the one or more signals.

12. (canceled)

13. The compliance monitoring system of claim 8 wherein the triangulation based measurement circuitry determines what locatee devices is closest to a dispenser.

14. The compliance monitoring system of claim 8 where at least one of the one or more locatee devices or associated with a non-stationary object.

15. The compliance monitoring system of claim 14 where the object is a person.

16. (canceled)

17. (canceled)

18. (canceled)

19. (canceled)

20. (canceled)

21. (canceled)

22. A dispenser for dispensing soap or sanitizer comprising:
a housing;
a processor;
a sensor for sensing an object;
a receptacle for receiving a container of soap or sanitizer;
a pump in fluid communication with the container;
an actuator for causing the pump to dispense a dose of fluid;
communication circuitry for wireless communication;
triangulation based locator circuitry for determining a location of one or more locatee devices;
two or more antennae for receiving signals from the one or more locatee devices; and
wake up circuitry for causing triangulation based locator circuitry to wake up and actively listen for signals from the one or more locatee devices and determine a location of the one or more locatee devices.

23. The dispenser of claim 21 wherein the triangulation based locator circuitry calculates an angle of arrival from one or more signals received by the two or more antennae.

24. The dispenser of claim 23 wherein the angle of arrival is a function of two or more measured times of flight values of the one or more signals.

25. The dispenser of claim 23 wherein the angle of arrival is a function of the difference between two or more timestamps of the one or more signals.

26. The dispenser of claim 23 wherein the angle of arrival is a function of phase differences of the one or more signals.

27. The compliance monitoring system of claim 21 wherein the triangulation based locator circuitry determines what locatee devices is closest to the dispenser.

28. (canceled)

29. (canceled)

30. (canceled)

31. (canceled)

32. (canceled)

33. (canceled)

34. (canceled)

35. (canceled)

36. (canceled)

37. (canceled)
